(19) United States
(12) Patent Application Publication
(10) Pub. No.: US 2018/0045978 A1
(43) Pub. Date: Feb. 15, 2018

(54) EYE LENS AND METHOD FOR MAKING SAME

(71) Applicant: HON HAI PRECISION INDUSTRY CO., LTD., New Taipei (TW)

(72) Inventor: HSIU-WEN CHIEN, New Taipei (TW)

(21) Appl. No.: 15/261,983

(22) Filed: Sep. 11, 2016

(30) Foreign Application Priority Data
Aug. 12, 2016 (TW) 105125869

(51) Int. Cl. G02C 7/04; G02B 1/04; B29C 35/08; A61K 31/352; B29D 11/00; C08K 5/1545; A61F 2/16

(52) U.S. Cl. CPC G02C 7/049; C08K 5/1545; G02B 1/043; A61F 2/1624; A61K 31/352; B29D 11/00038; B29D 11/00134; B29C 35/0805; A61F 2210/0061; A61F 2250/0067; B29C 2035/0827; B29K 2105/0061

(57) ABSTRACT

An eye lens carrying anthocyanins includes a matrix and a plurality of anthocyanins dispersed in the matrix. Since the colors of anthocyanins change depending on the PH of solution, acidic products released by microorganisms in the eye or eye lens will cause the colors of the anthocyanins to change from purple to pink, as an indicator to a user. Two methods for making a complete eye lens are also provided.

### FIELD

The subject matter generally relates to eye care.

### BACKGROUND

Eye lenses are widely used but are perfect environment for microorganisms to thrive, such as, bacteria, fungi, and other pathogens. When an eye lens is on a fingertip, the microorganisms begin to develop, and when the eye lens is put on the eye, eye infections probably occur if harmful microorganisms invade any part of the eyeball or surrounding area.

### BRIEF DESCRIPTION OF THE DRAWINGS

Implementations of the present technology will now be described, by way of example only, with reference to the attached figures.

FIG. 1 is a flow chart of a process for making an eye lens.

FIG. 2 is another flow chart of a process for making an eye lens.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

A definition that applies throughout this disclosure will now be presented.

The term “comprising” when utilized, means “including, but not necessarily limited to”; it specifically indicates open-ended inclusion or membership in the so-described combination, group, series, and the like.

An embodiment of an eye lens may be a contact lens or an intraocular lens (IOL). The intraocular lens may be an accommodating intraocular lens (AIOL). The eye lens includes a matrix and a plurality of anthocyanins dispersed in the matrix.

The matrix has a mass percentage of about 90% to about 99.99% of the total mass of the eye lens. The anthocyanins have a mass percentage of about 0.01% to about 10% of the total mass of the eye lens.

The eye lens has a PH of about 7, in other words, the eye lens is chemically neutral.

It is well known that anthocyanin is a derivative of benzopyran. The anthocyanin is a kind of water soluble flavonoids pigment, and the anthocyanin has antioxidant properties. Anthocyanins are widely found in most terrestrial plants, such as cactus, beet, onion, kidney bean, pomegranate and grape, and anthocyanins provide a color for these plants. The anthocyanins can change colors depending on the PH of solution: the anthocyanins are pink in acidic solutions (pH<7), purple in neutral solutions (pH=7), greenish-yellow in alkaline solutions (pH>7), and colourless in strong alkaline solutions.

Most of the microorganisms thrive in neutral environment, and release acidic products. Therefore, when there is no microorganisms in the eye lens, the anthocyanins are purple, the eye lens being purple too. When microorganisms thrive in the eye lens, the anthocyanins' color changes from purple to pink, thereby the eye lens' color changes from purple to pink too. Thereby, user can judge for himself whether the eye lens has been infected by microorganisms or not, and decide whether to wear the eye lens or not. This determination can avoid the eyes of the user being infected by microorganisms in the eye lens.

The anthocyanin also has anti-inflammatory, antiviral, and anticancer properties. When user wears the eye lens, anthocyanins can be released out from the eye lens, and apply its anti-inflammatory, antiviral, and anticancer properties.

The eye lens may be a hydrogel lens or a silicone hydrogel lens. When the eye lens is hydrogel lens, the matrix is hydrogel matrix. When the eye lens is silicone hydrogel lens, the matrix is silicone hydrogel matrix. The hydrogel matrix may be prepared by irradiating a hydrogel precursor with ultraviolet light (UV) to copolymerize the hydrogel precursor. The hydrogel precursor includes a hydrated polymer, a photoinitiator, and a cross-linking agent. The silicone hydrogel matrix may be prepared by irradiating a silicone hydrogel precursor with UV to copolymerize the silicone hydrogel precursor. The silicone hydrogel precursor includes a hydrated polymer, a photoinitiator, and a cross-linking agent.

When the matrix is a silicone hydrogel matrix, the hydrated polymer may be selected from a group consisting of methyl methacrylate (MMA) and hydroxyethyl methacrylate (HEMA). When the matrix is a silicone hydrogel matrix, the hydrated polymer may be selected from a group consisting of MMA, HEMA, polydimethylsiloxane (PDMS), and tris(hydroxymethyl)aminomethane (TRIS).

The photoinitiator may be obtained from Chemical Industries Basel (CIBA) Corporation as a clear liquid under the trade name “Irgacure-1173”. The cross-linking agent may be ethyleneglycol dimethacrylate (EGDMA).

The hydrogel precursor and silicone hydrogel precursor may further include a hydrophilic monomer. The hydrophilic monomer is configured to improve the water-affinity and oxygen permeability of the hydrogel matrix and the silicone hydrogel matrix. When the matrix is a hydrogel matrix, the hydrophilic monomer may be selected from a group consisting of N-Vinyl-2-pyrrolidone (NVP), glycidyl methacrylate (GMA), and N,N-Dimethylacrylamide (DMA). When the matrix is a silicone hydrogel matrix, the hydrophilic monomer may be N-Vinyl-2-pyrrolidone (NVP).

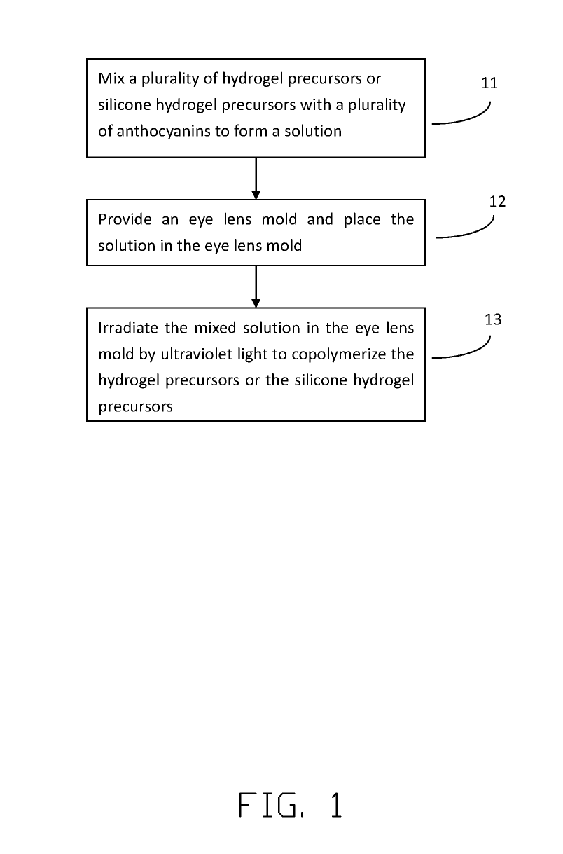

Referring to FIG. 1, a flowchart of a method for making the eye lens is presented. The example method is provided by way of example, as there are a variety of ways to carry out the method. The method can be carried out as illustrated in FIG. 1, for example. Each block shown in FIG. 1 represents one or more processes, methods, or subroutines carried out in the example method. Furthermore, the illustrated order of blocks is illustrative only and the order of the blocks can change. Additional blocks can be added or fewer blocks may be utilized without departing from this disclosure. The example method can begin at block 11.

At block 11, a plurality of hydrogel precursors or silicone hydrogel precursors are mixed with a plurality of anthocyanins, thereby a solution is formed.

At block 12, an eye lens mold is provided, and the solution is placed in the eye lens mold.

At block 13, the solution in the eye lens mold is irradiated by UV. The hydrogel precursor or the silicone hydrogel precursor is copolymerized to form a matrix with anthocyanins dispersed in it.

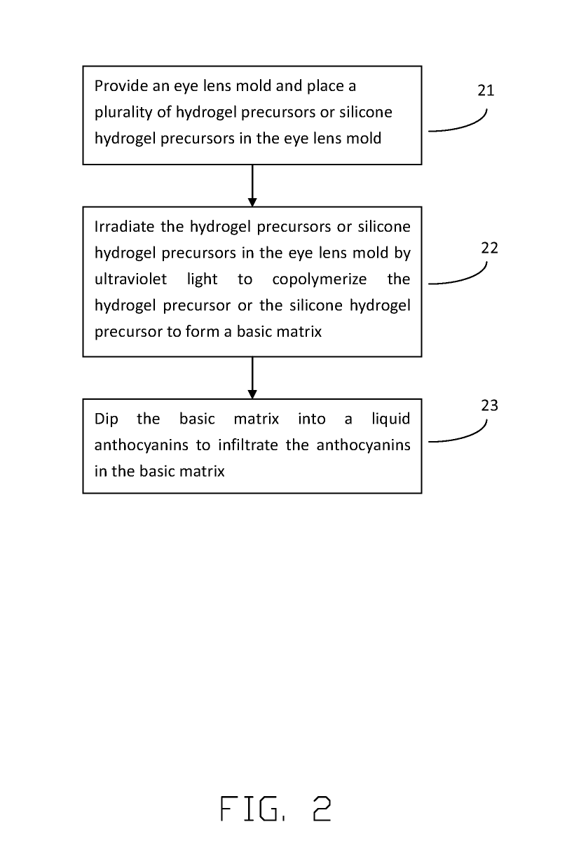

FIG. 2 shows a flowchart of another method for making the eye lens. The example method is provided by way of example, as there are a variety of ways to carry out the method. The method can be carried out as illustrated in FIG. 2. Each block shown in FIG. 2 represents one or more processes, methods, or subroutines carried out in the example method. Furthermore, the illustrated order of blocks is illustrative only and the order of the blocks can change. Additional blocks can be added or fewer blocks may be utilized without departing from this disclosure. The example method can begin at block 21.

At block 21, an eye lens mold is provided, and a plurality of hydrogel precursors or silicone hydrogel precursors are placed in the eye lens mold.

At block 22, the hydrogel precursor or the silicone hydrogel precursor in the eye lens mold are irradiated by UV, thereby the hydrogel precursor or silicone hydrogel precursor is copolymerized to form a basic matrix.

At block 23, the basic matrix is dipped into a liquid anthocyanins for a certain period of time to infuse the anthocyanins in the basic matrix.

### Example 1

The matrix of the eye lens was a hydrogel matrix. The matrix had a mass percentage of 98% of the total mass of the eye lens. The anthocyanins had a mass percentage of 2% of the total mass of the eye lens.

The eye lens was made by the following steps: (1) HEMA, EGDMA, Irgacure-1173, and anthocyanins were mixed, thereby a solution was formed. The HEMA had a mass percentage of 97.155% of the total mass of the solution, the Irgacure-1173 had a mass percentage of 0.545% of the total mass of the solution, the EGDMA had a mass percentage of 0.3% of the total mass of the solution, and the anthocyanins had a mass percentage of 2% of the total mass of the solution. (2) An eye lens mold was provided and the solution was placed in the eye lens mold. (3) The solution in the eye lens mold was irradiated by UV for 10 mins.

### Example 2

The matrix of the eye lens was a hydrogel matrix. The matrix had a mass percentage of 99.8% of the total mass of the eye lens. The anthocyanins had a mass percentage of 0.2% of the total mass of the eye lens.

The eye lens was made by the following steps: (1) an eye lens mold was provided, and HEMA, EGDMA, and Irgacure-1173 were mixed to form a hydrogel precursor. The hydrogel precursor was added into the eye lens mold, the HEMA had a mass percentage of 99% of the total mass of the hydrogel precursor, the Irgacure-1173 had a mass percentage of 0.58% of the total mass of the hydrogel precursor, the EGDMA had a mass percentage of 0.42% of the total mass of the hydrogel precursor. (2) The hydrogel precursor was irradiated by UV for 15 mins, thereby a basic matrix was formed. (3) The basic matrix was dipped into liquid state anthocyanins for 6 hours and then taken out, the anthocyanins had a mass concentration of 10%.

### Example 3

The matrix of the eye lens was a silicone hydrogel matrix. The matrix had a mass percentage of 98.35% of the total mass of the eye lens. The anthocyanins had a mass percentage of 1.65% of the total mass of the eye lens.

The eye lens was made by the following steps: (1) HEMA, DMA, TRIS, PDMS, Irgacure-1173, EGDMA, and anthocyanins were mixed, thereby a solution was formed. The HEMA had a mass percentage of 11.97% of the total mass of the solution, the DMA had a mass percentage of 33.6% of the total mass of the solution, the TRIS had a mass percentage of 49.5% of the total mass of the solution, the PDMS had a mass percentage of 1.3% of the total mass of the solution, the Irgacure-1173 had a mass percentage of 1.03% of the total mass of the solution, the EGDMA had a mass percentage of 0.95% of the total mass of the solution and the anthocyanins had a mass percentage of 1.65% of the total mass of the solution. (2) An eye lens mold was provided and the solution was placed in the eye lens mold. (3) The solution in the eye lens mold was irradiated by UV for 5 mins.

The embodiments shown and described above are only examples. Even though numerous characteristics and advantages of the present technology have been set forth in the foregoing description, together with details of the structures and function of the present disclosure, the disclosure is illustrative only, and changes can be made in the detail, including in matters of shape, size, and arrangement of the parts within the principles of the present disclosure, up to and including the full extent established by the broad general meaning of the terms used in the claims.

1. An eye lens comprising:
a matrix; and
a plurality of anthocyanins dispersed in the matrix, the anthocyanins have a mass percentage of about 2% of the total mass of the eye lens.

2. The eye lens of claim 1, wherein the matrix has a mass percentage of about 90% to about 99.99% of the total mass of the eye lens.

3. The eye lens of claim 1, wherein the eye lens has a PH of 7.

4. The eye lens of claim 1, wherein the matrix is a hydrogel matrix or a silicone hydrogel matrix, the hydrogel matrix is prepared by irradiating a hydrogel precursor with ultraviolet light to copolymerize the hydrogel precursor, the silicone hydrogel matrix is prepared by irradiating a silicone hydrogel precursor with ultraviolet light to copolymerize the silicone hydrogel precursor.

5. The eye lens of claim 4, wherein the hydrogel precursor or the silicone hydrogel precursor comprises a hydrated polymer, a photoinitiator, and a cross-linking agent.

6. The eye lens of claim 5, wherein the hydrogel precursor and silicone hydrogel precursor further comprises hydrophilic monomer, when the matrix is hydrogel matrix, the hydrophilic monomer is selected from a group consisting of N-Vinyl-2-pyrrolidone, glycidyl methacrylate, and N,N-Dimethylacrylamide; when the matrix is silicone hydrogel matrix, the hydrophilic monomer is N-Vinyl-2-pyrrolidone.

7. The eye lens of claim 5, wherein when the matrix is a silicone hydrogel matrix, the hydrated polymer is selected from a group consisting of methyl methacrylate and hydroxyethyl methacrylate; when the matrix is a silicone hydrogel matrix, the hydrated polymer is selected from a group consisting of methyl methacrylate, hydroxyethyl methacrylate, polydimethylsiloxane, and tris(hydroxymethyl)aminomethane.

8. The eye lens of claim 5, wherein the cross-linking agent is ethyleneglycol dimethacrylate.

9. A method for making an eye lens comprising:
mixing a plurality of hydrogel precursors or silicone hydrogel precursors with a plurality of anthocyanins to form a solution;
providing an eye lens mold and placing the solution in the eye lens mold; and
irradiating the mixed solution in the eye lens mold by ultraviolet light to copolymerize the hydrogel precursors or the silicone hydrogel precursors.

10. The method of claim 9, wherein the matrix has a mass percentage of about 90% to about 99.99% of the total mass of the eye lens, the anthocyanins have a mass percentage of about 0.01% to about 10% of the total mass of the eye lens.

11. The method of claim 9, wherein the matrix is a hydrogel matrix or a silicone hydrogel matrix, the hydrogel matrix is prepared by irradiating a hydrogel precursor with ultraviolet light to copolymerize the hydrogel precursor, the silicone hydrogel matrix is prepared by irradiating a silicone hydrogel precursor with ultraviolet light to copolymerize the silicone hydrogel precursor, the hydrogel precursor or the silicone hydrogel precursor comprises a hydrated polymer, a photoinitiator, and a cross-linking agent.

12. The method of claim 11, wherein the hydrogel precursor and silicone hydrogel precursor further comprises hydrophilic monomer, when the matrix is hydrogel matrix, the hydrophilic monomer is selected from a group consisting of N-Vinyl-2-pyrrolidone, glycidyl methacrylate, and N,N-Dimethylacrylamide; when the matrix is silicone hydrogel matrix, the hydrophilic monomer is N-Vinyl-2-pyrrolidone.

13. The method of claim 11, wherein when the matrix is a silicone hydrogel matrix, the hydrated polymer is selected from a group consisting of methyl methacrylate and hydroxyethyl methacrylate; when the matrix is a silicone hydrogel matrix, the hydrated polymer is selected from a group consisting of methyl methacrylate, hydroxyethyl methacrylate, polydimethylsiloxane, and tris(hydroxymethyl)aminomethane.

14. The method of claim 11, wherein the photoinitiator is Irgacure-1173, the cross-linking agent is ethyleneglycol dimethacrylate.

15. A method for making an eye lens comprising:
providing an eye lens mold and placing a plurality of hydrogel precursors or silicone hydrogel precursors in the eye lens mold;
irradiating the hydrogel precursors or silicone hydrogel precursors in the eye lens mold by ultraviolet light to copolymerize the hydrogel precursor or the silicone hydrogel precursor to form a basic matrix; and
dipping the basic matrix into a liquid anthocyanins to infiltrate the anthocyanins in the basic matrix.

16. The method of claim 15, wherein the matrix has a mass percentage of about 90% to about 99.99% of the total mass of the eye lens, the anthocyanins have a mass percentage of about 0.01% to about 10% of the total mass of the eye lens.

17. The method of claim 15, wherein the matrix is a hydrogel matrix or a silicone hydrogel matrix, the hydrogel matrix is prepared by irradiating a hydrogel precursor with ultraviolet light to copolymerize the hydrogel precursor, the silicone hydrogel matrix is prepared by irradiating a silicone hydrogel precursor with ultraviolet light to copolymerize the silicone hydrogel precursor, the hydrogel precursor or the silicone hydrogel precursor comprises a hydrated polymer, a photoinitiator, and a cross-linking agent.

18. The method of claim 17, wherein the hydrogel precursor and silicone hydrogel precursor further comprises hydrophilic monomer, when the matrix is hydrogel matrix, the hydrophilic monomer is selected from a group consisting of N-Vinyl-2-pyrrolidone, glycidyl methacrylate, and N,N-Dimethylacrylamide; when the matrix is silicone hydrogel matrix, the hydrophilic monomer is N-Vinyl-2-pyrrolidone.

19. The method of claim 17, wherein when the matrix is a silicone hydrogel matrix, the hydrated polymer is selected from a group consisting of methyl methacrylate and hydroxyethyl methacrylate; when the matrix is a silicone hydrogel matrix, the hydrated polymer is selected from a group consisting of methyl methacrylate, hydroxyethyl methacrylate, polydimethylsiloxane, and tris(hydroxymethyl)aminomethane.

20. The method of claim 17, wherein the photoinitiator is Irgacure-1173, the cross-linking agent is ethyleneglycol dimethacrylate.
